(19) United States
(12) Patent Application Publication
(10) Pub. No.: US 2016/0168315 A1
(43) Pub. Date: Jun. 16, 2016

(54) MODIFIED OLIGOESTERS AND MANUFACTURE METHODS OF THE OLIGOESTERS

(71) Applicant: ANHÍDRIDOS Y DERIVADOS DE COLOMBIA S.A. ANDERCOL, Medellin (CO)

(72) Inventors: Hugo Fernando HERNANDEZ GARCIA, Medellín (CO); Sebastian PAVONE CORREA, Medellín (CO); Juan David ILLERA QUINTERO, Medellín (CO)

(73) Assignee: ANHIDRIDOS Y DERIVADOS DE COLOMBIA S.A. ANDERCOL, Medellin (CO)

(21) Appl. No.: 14/908,558

(22) PCT Filed: Jul. 25, 2014
(86) PCT No.: PCT/CO2014/000009

§ 371 Date: Jan. 29, 2016

(30) Foreign Application Priority Data
Aug. 1, 2013 (CO) 13 182506

(51) Int. Cl. C08G 63/00; C08G 63/90; C09D 167/00; C08G 63/78; C07D 319/06; C07D 317/24

(52) U.S. Cl. CPC C08G 63/00; C07D 319/06; C07D 317/24; C09D 167/00; C08G 63/78; C08G 63/90

(57) ABSTRACT

The present invention relates to a modified oligoester including an oligoester (1) that in one of its ends has a group that reacts with alcohols or organic acids forming covalent bonds and in one or more of its other terminations has one or more cyclic acetal groups (2). In addition, relates to the processes for the production of such modified oligoester, as well as the use of the same in the manufacture of polyester resins, coating compositions, and composite, as well as the process for producing these resins. The scope of the present invention is related to the chemical sector, particularly in what refers to modified oligoesters with cyclic acetal groups.

### CROSS REFERENCE TO RELATED APPLICATION

This application is a national stage entry of PCT/CO2014/000009 filed Jul. 25, 2014, under the International Convention claiming priority over Colombian Patent Application No. 13182506 filed Aug. 1, 2013.

### TECHNICAL FIELD OF THE INVENTION

The field of application or technological sector of the present invention is related to the chemical sector, particularly in what refers to modified oligoesters with cyclic acetal groups, useful for the production of polyester resins, coating compositions, and composites.

### TECHNICAL PROBLEM

One of the most important features that a polyester resin should have is the low emission of dangerous air pollutants (HAPs) and/or volatile organic compounds (VOCs) during the curing or drying of the resin, an effect that is associated with the reduction of the content of the dilution solvent required to achieve the required resin viscosity. The content of resin solvent is considered to be low if its content is equal to or less than 35% by weight. In addition, the viscosity of the resin is considered low if it is equal to or less than 0.5 Pa·s/25° C. To achieve such an effect, it is necessary to obtain polymers of low molecular weight by the action of chain stoppers, a term that refers to chemical compounds capable of stopping the growth of a macromolecule, in at least one of its ends. A polyester resin is considered of low molecular weight when the average molecular weight is equal to or less than 2,000 Da. The chain stopper for the polyester resins needs to be capable of reacting with a single hydroxyl group or a single carboxyl group, located in one of the ends of the polymer, thereby preventing the polymer chain to continue to grow at that end.

Chain stoppers may be alcohols, monocarboxylic acids, or other types of molecules, the most representative being the dicyclopentadiene. This solution presents some difficulties: in the case of alcohols, if low molecular weight alcohols are used, there is an increase in the reaction times and additionally a low incorporation efficiency of the chain stopper, which means a significant increase in resin costs. If high molecular weight alcohols are used, there is no increase in reaction times and the incorporation efficiencies are high, but this affects the mechanical performance of the resins because of a plasticizing effect caused by the alcohols. In addition, high molecular weight alcohols promote inhibition by air during the curing of the resin.

Dicyclopentadiene (DCPD) has been the best alternative found for chain stopper so far, since it does not affect the reaction times, has good incorporation efficiencies in resin, and although it affects the mechanical performance of resins, its effect is minor compared with high molecular weight alcohols. However, the dicyclopentadiene is a compound that presents high risks for human health and for the environment, especially during the resin manufacturing process, since it is highly flammable (with a flash temperature of 26.7° C.), toxic (with an average lethal oral dose LD50 in rats of 353 mg/kg), of penetrating odor (with an odor threshold of 0.005 ppm), and disturbing (with an irritation threshold of 0.048 ppm). Additionally, the use of high amounts of dicyclopentadiene tends to produce side reactions during the process, generating very large molecular weight distributions which eventually increase the resin viscosity and therefore the required amount of solvent, causing a contrary effect to which it seeks to achieve. In regards to the polyester resins containing dicyclopentadiene, it has also shown a low chemical resistance especially in corrosive environments, as well as lower mechanical performance compared to the conventional resins that do not employ chain stoppers.

The objective of the present invention is to provide a chain stopper that does not increase the processing time of the polyester resins, which have high incorporation efficiencies, which does not affect the mechanical performance of resins, which avoid air inhibition during the curing, and in addition do not generate risks on human health or on the environment, in particular, during the resin manufacture.

### BACKGROUND OF THE INVENTION

Different types of chain stoppers for polyester resins have been documented previously. A polyester resin is disclosed in U.S. Pat. No. 6,268,464, with characteristics of high molecular weight (average molecular weight between 2000 and 6000 Da and the molecular weight average number between 700 and 2500, for the linear, unsaturated polyester of the invention), low viscosity (between 0.1 and 1 Pa·s/125° C., preferably between 0.3 and 0.8 Pa·s/125° C.) and high solubility in reactive solvents (between 20 and 35% by weight), where the resin is obtained by the reaction between polycarboxylic acid, with a diol and the action of a monoalcohol as a chain stopper. The production of this type of resins presents a difficulty since low molecular weight alcohols have very low boiling points in comparison to the rest of the components, which reduces the efficiency of the chain stopper in the resin and increases the reaction times, and thus the costs of the process. On the other hand the high molecular weight alcohols have boiling points equal or superior to the other components but they incorporate a plasticizing effect in the polymer that negatively impacts their mechanical performance, in addition these resins are susceptible to air inhibition during the curing, providing an undesirable sticky surface.

U.S. Pat. No. 6,794,483 discloses a resin with low styrene emissions (less than 35% by weight) that reduces the required amount of alcohol making the process more economical, this is accomplished through the implementation of small monohydric alcohols and dicyclopentadiene (DCPD) as chain stoppers. The combination of these chain stoppers improves the mechanical properties of the resin, but the incorporation of dicyclopentadiene generates risks for health and the environment, especially during the resin manufacturing process, since this is a highly flammable compound, toxic, with a penetrating and annoying smell, which presents high risks of contamination. The main risks of the dicyclopentadiene have been widely documented (NIOSH, 1995; UNEP OECD SIDS, 1998). Additionally the dicyclopentadiene tends to produce side reactions during the process, generating very large molecular weight distributions which eventually increase the viscosity of resin and therefore the amount of solvent needed. As for the polyester resins containing dicyclopentadiene, it has shown a low chemical resistance especially in corrosive environments, as well as lower mechanical performance than conventional resins.

The use of acetals as chain stoppers has also been previously documented. A polyester resin that can be cured with air without presenting inhibition is disclosed on U.S. Pat. No. 3,210,441, which incorporates within its process and structure, monohydric cyclic acetals as chain stoppers, where such resin is produced by the reaction of polycarboxylic acids and diols. This type of product requires the addition of dicyclopentadiene to decrease the viscosity of the resin, incorporating all the problems and drawbacks listed above.

U.S. Pat. No. 3,291,860 disclosed a curing agent that improves the serving life of the polyester resins, which is constituted by a cyclic acetal, which does not meet the chain stopper function, which generated polymers of high molecular weight with a resulting high viscosity, which require a high amount of reagent solvent to make the product function. The main disadvantage of the incorporation of cyclic acetals directly on polyester resin is that carboxylic acids used in the manufacture of the resin, catalyze a series of side reactions in the acetal which lead to the formation of branches in the polyester, which increase the viscosity of the resin, produce gelling, and incompatibility with the styrene.

U.S. Pat. No. 6,927,275 discloses a procedure to produce polyester resins with good mechanical properties, including dicarboxylic acids and diols, some of which have a cyclic acetal skeleton, solving the disadvantages of the use of cyclic acetals as indicated above. In this developing, the cyclic acetals are not incorporated as chain stoppers, which generates high molecular weight polymers with a resulting high viscosity, which require a high amount of solvent reactive to make the functional product.

### SUMMARY OF THE INVENTION

The present invention relates to a modified oligoester comprising an oligoester (1) containing a group which forms covalent bonds with —OH or —COOH groups in their terminations and that is bound in one or more of the ends of its chain with cyclic acetal groups (2).

In an embodiment of the invention, the modified oligoester is characterized because the oligoester (1) comes from the esterification reaction between esterifiable monomers selected from the group consisting of polyols (3), carboxylic polyacids, and hydroxy acids (5), or

depolymerization of virgin or waste polyesters, including but not limited to the following:

polyglycolic acid, polylactic acid, polycaprolactone, polyethylene adipate, polyethylene naftalato, polyethylene terephthalate, polybutylene succinate, polybutylene terephthalate, polytrimethylene terephthalate, polyhydroxybutyrate, polyhydroxyvalerate.

Another embodiment of the present invention refers to a process for obtaining the previously described modified oligoester which includes steps of:

the synthesis of an oligoester (1) via the depolymerization of polyesters or the reaction between esterifiables monomers selected from the group consisting of polyols (3), carboxylic polyacids (4), and hydroxy acids (5),

the reaction between the oligoester of the step (a) and a compound containing one or more cyclic acetals groups and one or more alcohol groups.

Another embodiment of the present invention refers to a chemical composition characterized because it contains the modified oligoester previously described and at least one compound selected from the group formed by catalysts (19), solvents (20), and (21) additives.

Another embodiment of the present invention refers to a polyester resin containing a polyester synthesized with the modified oligoester as previously described.

In an embodiment of the invention, the polyester resin is characterized because it does not contain dicyclopentadiene.

In another embodiment of the invention, the polyester resin is characterized because the styrene content as solvent is between 0% and 35% in weight.

In another embodiment of the invention, the polyester resin is characterized by a content of compounds classified as HAPs equal or less than 35% by weight.

In another embodiment of the invention, the polyester resin is characterized by a content of compounds classified as VOCs equal to or less than 35% by weight.

Another embodiment of the present invention refers to a process for obtaining the polyester resin as previously described characterized because it includes the following steps:

the polymerization in the presence of the previously described modified oligoester, of two or more monomers selected from the group comprising hydroxyacids (5), polyols (3), carboxylic polyacids (4),

adjusting the polyester obtained in step (a).

Another aspect of the present invention refers to a coating composition characterized because it contains a polyester resin as previously described as film-forming.

Another aspect of the present invention refers to a composite characterized because it contains the polyester resin as previously described as a polymeric matrix.

Another aspect of the present invention refers to the use of the modified oligoester as previously described as a chain stopper for the manufacture of polyester resins.

### DETAILED DESCRIPTION OF THE INVENTION

The modified oligoester of the present invention provides a chain stopper for polyesterification processes, which allows obtaining low-molecular-weight polymers which require lower solvent content for its dilution, without increasing the process time for the polyester resins, presenting high incorporation efficiency of the resin, avoiding air inhibition during the curing, without generating risks on the human health or on the environment, especially during the resin manufacture, and without significantly affecting the chemical and mechanical resistance of the resin, as happens with the chain stoppers employed in the state of the art.

Oligoester refers to an oligomer whose monomer units are connected by means of ester type links.

Oligomer refers to molecular structures containing two or more ester links between basic building blocks, without going beyond ten ester links.

Polyesterification refers to the process of polymerization that involves both esterification and transesterification reactions.

Polymerization refers to the process through which macromolecules of higher degree of polymerization are built by the incorporation of additional monomer units.

Macromolecule refers to a molecular structure including 2 or more monomer units. A macromolecule may be an oligomer or a polymer depending on the degree of polymerization.

Degree of polymerization refers to the number of monomer units that make up a macromolecule.

Esterification refers to the process by which a carboxyl group reacts with a hydroxyl group, forming an ester type bond.

Transesterification refers to the process by which the ester group of a molecule is reacted with the carboxyl group or the hydroxyl group of another, forming a new ester bond between the two molecules. As a by-product of the reaction, a molecule is released containing a carboxyl group or a hydroxyl group respectively, different to the one that originally participated in the reaction.

The oligoester of the present invention is a non-toxic material, is not flammable, and does not present other adverse effects on the environment and human health.

Toxic material refers to chemical compounds showing an average oral lethal dose (LD50) in rats of less than 500 mg/kg.

Flammable material refers to chemical compounds that have a flask point of less than 37.8° C. (100° F.).

The oligoester proposed by the present invention includes an oligoester which contains cyclic acetal groups covalently linked to one or more of the ends of the chain.

Cyclic acetal group refers to a part of a molecule that includes a ring or cycle, which is characterized by containing one or more acetal type links (—O—C—O—).

The modification of oligoesters by cyclic acetal groups is made by the reaction of one of the ends of the oligoester with a compound with cyclic acetal groups

Some non-limiting examples of these modification reactions include:

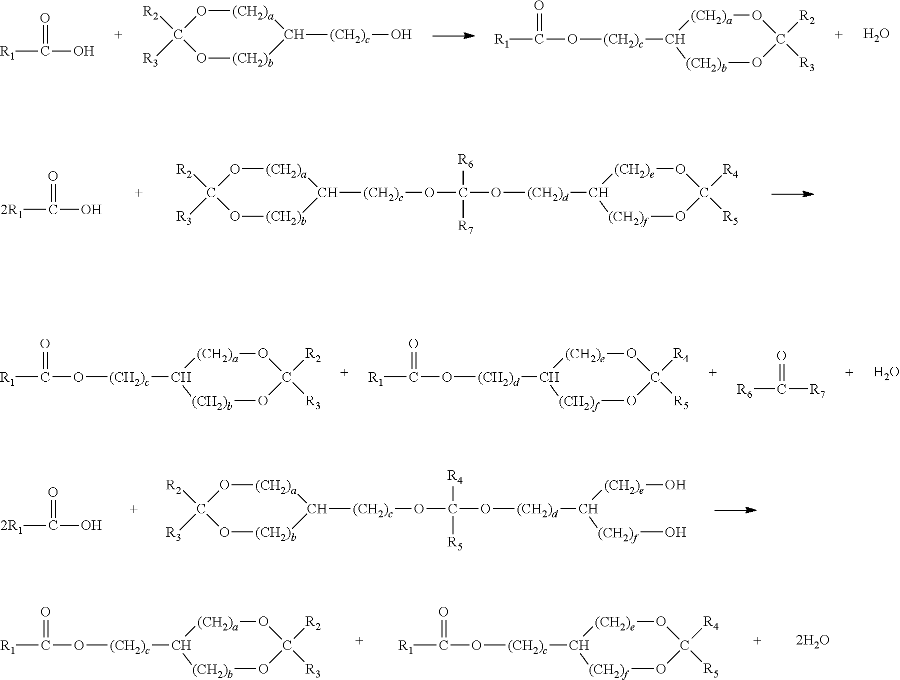

where R₁ represents a growing oligoester, R₂-R₇ represent organic groups in general (including hydrogens), and a, b, c, d, e, f are positive whole numbers (greater or equal to zero).

Growing oligoester refers to one in which one of its ends or terminations includes a reactive group that allows the formation of covalent bonds with alcohols, acids and esters during a polyesterification process. The reactive groups in the terminations of a growing oligoester include but are not limited to —OH and —COOH.

The modified oligoester of the present invention is obtained starting from an oligoester which may or may not have modifications at their ends, wherein the ends contains a reactive group capable of forming covalent bonds with alcohol or organic acid groups, where that group is selected from the group including alcohols, monocarboxylic acids, aldehydes, ketones, amines, acyl halides, inorganic acids, and hydroxides.

Alcohol refers to molecules containing only a hydroxy group at its structure, and does not contain any acid group or carboxylic anhydride.

Examples of alcohols include, but are not limited to, 2-ethyl hexanol, 2-hydroxy-1,4-benzoquinone, 2-hydroxy-1,4-naphthoquinone, 2-methylaminoethanol, 3-Hydroxy-1,2-naphthoquinone, 4-hydroxybenzophenone, 4-hydroxymethyl-1,3-dioxolane, 4-(hydroxymethyl)-1,3-dioxolane-2-one, 4-(hydroxymethyl)pyridine, 5-hydroxy-1,3-dioxane, 5-hydroxy-1,4-naphthoquinone, 5-(hydroxymethyl)-2-furaldehyde, allyl alcohol, amyl alcohol, benzyl alcohol, cetyl alcohol, stearyl alcohol, lauric alcohol, butanol, cyclohexanol, decanol, ethanol, ethanolamine, eugenol, phenol, geraniol, isopropanol, isopropylidene glycerol, menthol, methanol, nonanol, octanol, propanol, tridecanol, undecanol, and vanillin.

Monocarboxylic acid refers to a molecule containing only one carboxylic acid group in it structure, and does not contain any hydroxyl group.

Examples of monocarboxylic acids include, but are not limited to, acetic acid, benzoic acid, butyric acid, capric acid, caprylic acid, caproic acid, chloroacetic acid, stearic acid, formic acid, lauric acid, myristic acid, palmitic acid, pyruvic acid, propionic acid, and valeric acid.

Aldehyde refers to a molecule that contains one or more aldehyde groups, but does not contain hydroxy groups, or acid, or anhydride carboxylic groups in its structure.

Examples of aldehydes include, but are not limited to, acetaldehyde, benzaldehyde, butyraldehyde, cinnamaldehyde, formaldehyde, phthalaldehyde, furfural, Glyoxal, glutaraldehyde, propionaldehyde, retinaldehyde, succinaldehyde, tolualdehyde, and valeraldehyde.

Ketone refers to a molecule that contains one or more ketone groups, but does not contain hydroxyl groups or acid, or carboxylic anhydride groups in its structure.

Examples of ketones include, but are not limited to, acetone, acetophenone, benzophenone, cyclohexanone, cyclopropanone, isophorone, methyl ethyl ketone, methyl isobutyl ketone, and methyl vinyl ketone.

Amine refers to a molecule that contains one or more amino groups, but does not include hydroxyl groups, or acid, or carboxylic anhydride groups in its structure.

Examples of amines include, but are not limited to, 2-aminopentanol, 2-propylamine, 4-methylaniline, ammonia, aniline, aziridine, diphenylamine, dimethylamine, hydrazine, methylamine, piperidine, propylamine, triphenylamine, and trimethylamine.

Acyl halide refers to a molecule that contains one or more groups of acyl halide, but not hydroxyl groups, or acid, or carboxylic anhydride in its structure.

Examples of acyl halides include, but are not limited to, acetyl bromide, acetyl chloride, acetyl iodide, acryloyl chloride, adipoyl chloride, benzoyl chloride, chloroacetyl chloride, fluoroacetyl chloride, formyl fluoride, methacryloyl chloride, methoxybenzoyl chloride, oxalyl chloride, and oxalyl fluoride.

Inorganic acid refers to a molecule that contains one or more inorganic acid groups or minerals, but does not include hydroxyl groups, or acid, or carboxylic anhydride groups in its structure.

Examples of inorganic acids include, but are not limited to, boric acid, hydrobromic acid, hydrochloric acid, hydrofluoric acid, phosphoric acid, nitric acid, perchloric acid, and sulfuric acid.

Hydroxide refers to a molecule that contains one or more inorganic hydroxyl groups, but does not contain hydroxyl groups, or acid, or carboxylic anhydride groups in its structure.

Examples of hydroxides include, but are not limited to, aluminum hydroxide, barium hydroxide, calcium hydroxide, lithium hydroxide, magnesium hydroxide, potassium hydroxide, and sodium hydroxide.

The oligoester according to the present invention can be obtained through the polymerization of esterifiable monomers, or through polyesters depolymerization.

Esterifiable monomer refers to basic molecules capable of reacting in 2 or more of their ends forming ester type bonds. Non-limiting examples of esterifiable monomers include polyols, carboxylic poly acids, and hydroxyacids.

Polyol refers to molecules with two or more hydroxyl groups in its structure. Polyols can also be referred to as polyhydric alcohols. When the molecule has two hydroxyl groups, it is often called diol or glycol. When the molecule has three hydroxyl groups, it is often called triol.

Examples of polyols include, but are not limited to 1,2-ethanediol, 1,1,2-propanediol, 1,2,3-propanetriol, 1,2,3-benzenetriol, 1,2,3,4-tetraol, 1,2,3,4,5-pentol, 1,2,3,4,5,6-hexol, 1,2,4-bencenotriol, 1,2,6,1,3-propanediol, 1,3,5-bencenotriol, 1,4-butanediol, 1,4-cyclohexane dimethanol, 1,4-cyclohexanediol, 1,4-dianhydro-D-sorbitol, 1,5-pentanediol, 1,6-hexanediol, 1,7-heptanediol, 1,8-octanediol, 1,9-nonanediol, 1,10-decanediol, 2-amino-2-hydroxymethyl-propane-1,3-diol, 2,2-bis(hydroxymethyl)-1,3-propanediol, 2,2-dimethyl-1,3-propanediol, 2,2-diethyl-1,3-propanediol, 2-(2-hydroxy-1-methylethoxy)propanol, 2-(2-hydroxy-propoxy)propanol, 2-[2-(2-hydroxyethoxy)ethanol, 2,2-bis(4-hydroxyphenyl)butane, 2,2-bis(4-hydroxyphenyl)propane, 2-2′-(oxy bis(ethylene oxy)di-ethanol, 2,3,4,5,6-pentahydroxyhexanal, 2-butyl-2-ethyl-1,3-propanediol, 2-(hydroxymethyl)-2-methylpropane-1,3-diol, 2-(hydroxymethyl)-2-ethylpropane-1,3-diol, 2-(hydroxyethoxy)ethanol, 2-ethyl-1,4-butanediol, 2-methyl-1,3-propanediol, 2-methyl-1,4-butanediol, 3-methyl-1,5-pentanediol, 3-methyl-1,5-heptanediol, (3S)-2-(1,2-dihydroxyethyl)tetrahydrofuran-3,4-diol, 4-(hydroxymethyl)phenol, 4-oxa-2,6-heptanediol, 4-O-α-glucopyranosyl-D-sorbitol, 9-octadecenoic acid-12-hydroxy-1,1′,1″-(1,2,3-propanetriyl)ester, bis(4-hydroxyphenyl)methane, tris(2-hydroxyethyl)amine, and α-D-glucopyranosyl.

Carboxylic polyacids refer to molecules with two or more carboxylic acid groups, or one or more carboxylic anhydride groups in its structure. Molecules with two carboxyl groups, or a carboxylic acid anhydride group is commonly called dibasic acid or diacid.

Examples of carboxylic polyacids include, but are not limited to 1,1-cyclobutyl dicarboxylic acid, 1,2-cyclohexyl dicarboxylic acid, 1,3-cyclohexyl dicarboxylic acid, 1,4-cyclohexyl dicarboxylic acid, aconitic acid, adipic acid, azelaic acid, citraconic acid, dihydromuconic acid, dodecanedioic acid, phthalic acid, glutaconic acid, glutaric acid, isophthalic acid, itaconic acid, maleic acid, malonic acid, methylic acid, mesaconic acid, methylcyclohexane-1,2-dicarboxylic acid, 2-methylglutaric acid, muconic acid, oxalic acid, pimelic acid, pyromellitic acid, propane-1,2,3-tricarboxylic acid, sebacic acid, suberic acid, succinic acid, terephthalic acid, traumatic acid, trimellitic acid, trimesic acid, undecanedioic acid, 1,2-cyclohexyl dicarboxylic anhydride, phthalic anhydride, maleic anhydride, methylcyclohexane-1,2-dicarboxylic anhydride, succinic anhydride, and naphthalenetetracarboxylic dianhydride.

Hydroxyacid refers to a molecule containing one or more hydroxyl groups in its structure and one or more carboxylic acid groups.

Examples of hydroxyacids include, but are not limited to, 3-hydroxybenzoic acid, 3-hydroxypropanoic acid, 4-hydroxybenzoic acid, 4-(hydroxymethyl)benzoic acid, 6-hydroxyadipic acid, 16-hydroxypalmitic acid, 18-hydroxyestearic acid, aldaric acid, glyceric acid, citric acid, glycolic acid, isocitric acid, lactic acid, malic acid, mandelic acid, salicylic acid, tartaric acid, β-hydroxybutyric acid, and β-hydroxy-β-methylbutyric acid.

Depolymerization refers to the process by which macromolecules are broken down, resulting in the formation of molecular structures of smaller size (monomer units or macromolecules of lesser degree of polymerization).

Polymer refers to a molecular structure composed of more than ten monomer units.

Polyester means a polymer whose monomer units are linked by means of ester type bonds.

Examples of polyesters include, but are not limited to, polyglycolic acid, polylactic acid, polycaprolactone, polyethylene adipate, polyethylene naphthalate, polyethylene terephthalate, polybutylene succinate, polybutylene terephthalate, polytrimethylene terephthalate, polyhydroxybutyrate, polyhydroxyvalerate.

Polyesters used to obtain the oligoesters using the depolymerization technique may be virgin polyesters or waste polyesters.

Virgin polyester refers to polyester that has not had any application after having been manufactured.

Waste polyester refers to polyester that has already completed its life cycle in a previous application.

Cyclic acetals groups used to modify the oligoester of the present invention may come from monohydric cyclic acetals, preferably from the reaction of a triol (preferably glycerol) and an aldehyde or ketone, or can also come from oligoacetals.

Monohydric cyclic acetal refers to a molecule that contains in its structure a cyclic acetal group and a hydroxyl group. It may also be referred to as monohydric cyclic acetal.

Examples of monohydric cyclic acetals include, but are not limited to, 2,2-dimethyl-4-hydroxybutyl-1,3-dioxolane, 2,2-dimethyl-4-hydroxymethyl-1,3-dioxolane, 2,2-dimethyl-5-hydroxy-1,3-dioxonane, 2-(4-methylphenyl)-4-hydroxybutyl-1,3-dioxolane, 2-(4-methylphenyl)-4-hydroxymethyl-1,3-dioxolane, 2-(4-methylphenyl)-5-hydroxy-1,3-dioxane, 2-(4-methylphenyl)-5-hydroxy-1,3-dioxonane, 2-ethyl-4-hydroxybutyl-1,3-dioxolane, 2-ethyl-4-hydroxymethyl-1,3-dioxolane, 2-ethyl-4-hydroxy-1,3-benzodioxol, 2-ethyl-5-hydroxy-1,3-benzodioxol, 2-ethyl-5-hydroxy-1,3-dioxane, 2-ethyl-5-hydroxy-1,3-dioxonane, 2-phenyl-4-hydroxybutyl-1,3-dioxolane, 2-phenyl-4-hydroxymethyl-1,3-dioxolane, 2-phenyl-5-hydroxy-1,3-benzodioxol, 2-phenyl-5-hydroxy-1,3-dioxane, 2-phenyl-5-hydroxy-1,3-dioxonane, 2-(furan-2-yl)-4-hydroxybutyl-1,3-dioxolane, 2-(furan-2-yl)-4-hydroxymethyl-1,3-dioxolane, 2-(furan-2-yl)-5-hydroxy-1,3-dioxane, 2-(furan-2-yl)-5-hydroxy-1,3-dioxonane, 2-methyl-4-hydroxybutyl-1,3-dioxolane, 2-methyl-4-hydroxymethyl-1,3-dioxolane, 2-methyl-4-hydroxy-1,3-benzodioxol, 2-methyl-5-hydroxy-1,3-benzodioxol, 2-methyl-5-hydroxy-1,3-dioxane, 2-methyl-5-hydroxy-1,3-dioxonane, 2-propyl-4-hydroxybutyl-1,3-dioxolane, 2-propyl-4-hydroxymethyl-1,3-dioxolane, 2-propyl-5-hydroxy-1,3-benzodioxol, 2-propyl-5-hydroxy-1,3-dioxane, 2-propyl-5-hydroxy-1,3-dioxonane 4-(1,3-dioxan-2-yl)-2-methoxyphenol, 4-(1,3-dioxepan-2-yl)-2-methoxyphenol, 4-(1,3-dioxalane-2-yl)-2-methoxyphenol, 4-hydroxybutyl-1,3-dioxolane, 4-hydroxymethyl-1,3-dioxolane, 4-hydroxy-1,3-benzodioxol, 5-hydroxy-1,3-benzodioxol, 5-hydroxy-1,3-dioxane, and 5-hydroxy-1,3-dioxonane.

Glycerol refers to all compositions that mostly contain 1,2,3-propanetriol, which include, but are not limited to, USP grade glycerine, technical grade glycerine, crude glycerine byproduct of the process of manufacture of soap, and crude glycerine byproduct of the biodiesel production process.

Oligoacetal refers to oligomer composed of two or more hydroxylated compounds, may be monohydric or polyhydric, joined by an acetal type bound. The oligoacetals may be obtained, but without being limited to, by the reaction of one or more polyols with one or more aldehydes and/or one or more ketones.

The compounds used as a source of cyclic acetals in the present invention are those derived preferably from one or more triols with one or more aldehydes and/or one or more ketones. Among the triols employed to obtain the compounds used as a source of cyclic acetal groups, glycerol is preferable, which when it is derived from naturally occurring triglycerides, contributes to the renewable character of the modified oligoester of the present invention. In obtaining compounds used as a source of cyclic acetals, a relation of hydroxyl to carbonyl equivalents between 1 and 5, preferably between 2 and 4, and most preferably between 2.5 and 3.5 is used.

The modified oligoesters with cyclic acetals of the present invention may be used to obtain a chemical composition that contains the modified oligoester and one or more components selected from the group consisting of catalysts, solvents, and additives.

Catalyst refers to all compounds used as raw material in any proportion in a reaction in order to speed up the reaction.

Examples of catalysts include, but are not limited to, acids or inorganic anhydrides, acid or organic anhydrides, organometallic acids, metallic carboxylates, carboxylate organometallic, enzymes, metal hydroxides, metal oxides, organometallic oxides, ion exchange resins, and zeolites.

The amount of catalyst employed in the chemical composition may be between about 0.001% to about 2% of the chemical composition; preferably between about 0.01% to about 0.5% of the chemical composition; and more preferably between about 0.01% to about 0.1% of the chemical composition.

Solvent refers to a fluid able to dilute, dissolve, or disperse, one or more compounds or products of interest.

Examples of solvents include, but are not limited to, water, alcohols, aldehydes, ketones, esters, ethers, aliphatic hydrocarbons, aromatic hydrocarbons, halogenated hydrocarbons, polyols, or mixtures of them.

The amount of solvent employed in the chemical composition may be between about 0.1% to about 70% of the chemical composition; preferably between about 1% to about 50% of the chemical composition; and more preferably between about 5% to about 35% of the chemical composition.

Additive refers to any compound used as raw material in a proportion equal to or less than 2% by weight of the final composition, in order to modify the properties or functions of the composition. Additives may include, but are not limited to, antifoaming, air release agents, thixotropic agents, biocides, compatibilizers, dispersants, stabilizers, moisturizers, inhibitors, pigments and dyes, promoters and co-promoters, fillers, flame retardants, suppressors, and surfactants.

The amount of additive employed in the chemical composition may be between about 0.001% to about 2% of the chemical composition; preferably between about 0.005% to about 0.5% of the chemical composition; and more preferably between about 0.005% to about 0.1% of the chemical composition.

The oligoesters that can be modified with cyclic acetals groups according to the present invention, can be obtained through two main processes: esterification or transesterification.

The process of esterification for the obtaining of the modified oligoester of the present invention includes the steps of (a) charging the raw materials, (b) adjusting the process conditions, and (c) esterification. The raw materials employed in this process may be polyols, polycarboxylic acids, hydroxyacids, monohydroxy cyclic acetals, oligoacetals, catalysts, solvents, and additives.

Raw materials refer to those compounds and materials used in a process to obtain a product.

Process conditions refer to the state of a system required to carry out a step of the process, and characterized in this case by the pressure and the temperature.

In the esterification process, the oligoesters are obtained from the esterification reaction of polyol monomers with carboxylic polyacids, and/or by the esterification reaction of hydroxyacids monomers. Additionally, the oligoesters may be modified at one end by a molecule capable of reacting with hydroxyl groups or with the carboxyl groups, such as alcohols, acids, aldehydes, ketones, amines, monocarboxylic, acyl halides, inorganic acids, and hydroxides.

In obtaining oligoesters by the esterification process a relationship of hydroxyl to carboxyl equivalents between 1.1 and 1.5, preferably between 1.12 and 1.25, and most preferably between 1.2 and 1.25 is used. To carry out this esterification reaction a reaction temperature between 50° C. and 350° C., preferably between 100° C. and 300° C., and more preferably between 150° C. and 250° C. is employed. It is used additionally, a pressure reaction usually between 10 kPa and 2 MPa, preferably between 20 kPa and 1 MPa, and most preferably between 50 kPa and 0.5 MPa.

As catalyst of the esterification reaction can be used organic or inorganic acids or anhydrides, organometallic acids, metal carboxylates, organometallic carboxylates, metal hydroxides, lipases, organometallic oxides, acidic or basic ion exchange resins, acidic or basic zeolites, among others. Preferably it may use as catalyst inorganic acids compounds, organometallic acids, acidic ion exchange resins or acidic zeolites. Most preferably it may be used as catalyst inorganic acids and organometallic acids.

The amount of catalyst employed in the esterification is usually lower than 2% by weight of the total of the reagents, preferably less than 0.5% by weight of the total of the reagents, and more preferably between 0.01% and 0.1% by weight of the total of the reagents. Esterification reaction progresses until an average degree of polymerization preferably between 2 and 10 is reached. In addition, the esterification process may include a step of purification of the oligoester.

Non-limiting examples of catalysts for esterification include 2-ethylhexyl titanate, butyl stannoic acid, phosphoric acid, nitric acid, methanesulfonic acid, perchloric acid, p-toluenesulfonic acid, sulfuric acid, butyl isopropyl titanate, dibutyltin diacetate, butylchlorotin dihydroxide, dibutyltin dilaurate, Aspergillus niger lipase, Candida antarctica lipase A, Candida antarctica lipase B, Rhizopus arrhizus lipase, dibutylstannous oxide, dioctylstannous oxide, monobutylstannous oxide, polystyrene resins modified with sulphate or sulphonate groups, tetrabutyltin, tetrabutyl titanate, tetraisopropyl titanate, butyltin tris(2-ethyl hexanoate).

Purification refers to the set of procedures and operations performed in a process in order to increase the purity or concentration of a compound or product. Purification may refer, without limiting, to one or more of the following operations: absorption, crystallization, desorption, distillation, evaporation, extraction, filtration, and drying.

Example 1 presents the synthesis of modified oligoesters with cyclic acetals using the esterification process between a polyol and a carboxylic anhydride. As a source of cyclic acetals, a monohydric cyclic acetal is used.

The process of transesterification to obtain the modified oligoester of the present invention includes the steps of (a) charging the raw materials, (b) adequacy of the process conditions, and (c) transesterification. The raw materials employed in this process may be polyesters, monohydric cyclic acetals, oligoacetals, polyols, catalysts, solvents, and additives.

In the process of transesterification, the oligoesters are obtained from reactions of substitution between low molecular weight compounds that contain hydroxyl groups or carboxyl groups and polyesters. Preferably, the low molecular weight compounds containing hydroxyl groups and cyclic acetals groups in its structure. These substitution reactions with low molecular weight compounds lead to the reduction of the polymerization degree of the polymer chains. The polyesters used may be waste polyesters, which contribute to increasing the content of recycled material in the oligoester.

In obtaining oligoesters using the process of transesterification a relation of hydroxyl to monoester equivalents usually between 0.5 and 3, preferably between 0.75 and 2.5, and more preferably between 1 and 2, is used. To carry out this transesterification reaction, a temperature between 50° C. and 350° C., preferably between 100° C. and 300° C., and more preferably between 150° C. and 250° C. is employed. Additionally a reaction pressure usually between 10 kPa and 2 MPa, preferably between 20 kPa and 1 MPa, and most preferably between 50 kPa and 0.5 MPa is used.

As catalyst for the transesterification reaction may be used organic or inorganic acids or anhydrides, organometallic acids, metallic carboxylates, organometallic carboxylate, metallic hydroxides, lipases, organometallic oxides, among others. Preferably, the catalyst may be inorganic acid compounds, organometallic acids, and metallic carboxylates.

The amount of the esterification catalyst is usually lower than 2% by weight of the total of the reagents, preferably less than 0.5%, and more preferably between 0.01% and 0.1%. The transesterification reaction is carried out until an average degree of polymerization between 2 and 10 is reached. Additionally, the process of transesterification may include a step of purification of the modified oligoester.

Non-limiting examples of transesterification catalyst include 2-ethylhexyl titanate, tin acetate, magnesium acetate, manganese acetate, zinc acetate, butyl stannoic acid, phosphoric acid, methanesulfonic acid, p-toluene sulfonic acid, sulfuric acid, tin borohydride, potasium borohydride, sodium borohydride, titanium borohydride, butyl isopropyl titanate, lithium carbonate, magnesium carbonate, sodium carbonate, dibutyltin diacetate, dibutyltin dilaurate, antimony glycoxide, magnesium hydroxide, Aspergillus niger lipase, Candida antarctica lipase A, Candida antarctica lipase B, Rhizopus arrhizus lipase, tin oxide, magnesium oxide, dibutylstannous oxide, dioctylstannous oxide, monobutylstannous oxide, tetraisopropyl titanate, tetrabutyl titanate, antimony trioxide, and tetrabutyltin.

Examples 2 to 5 present modified oligoesters with cyclic acetals obtained by the transesterification process. Examples 2 and 4 use waste polyester, Example 3 uses virgin polyester and Example 5 uses a mixture of virgin polyester with waste polyester. Example 2 uses oligoacetals as source of cyclic acetals, while in Examples 3 through 5 monohydric cyclic acetals are used as source of cyclic acetals.

The oligoesters modified with cyclic acetals of the present invention are especially useful as chain stoppers in the synthesis of polyester resins, in particular of low viscosity polyester resins using a low content of solvents, that may be used for the manufacture of polymeric materials of low volatile organic compounds (VOC) or hazardous air pollutants (HAPs) emissions, and less odor.

The amount of chain stopper employed in the chemical composition may be between about 0.01% to about 80% of the chemical composition; preferably between about 5% to about 65% of the chemical composition; and more preferably between about 20% to about 50% of the chemical composition.

Chain stopper refers to a chemical compound capable of stopping the growth of a macromolecule, in at least one of its ends.

Polyester resin refers to a polyester composition with solvent, which may belong, but not be limited to, one of the following groups: alkyd resins, unsaturated polyester resins, and saturated polyester resins.

Low viscosity refers to a viscosity of the polyester resin less or equal than 0.5 Pa·s/25° C.

Low solvent content refers to a content equal to or below 35% by weight of compounds used as solvents.

HAP or HAPs refers to those compounds that are classified as hazardous air pollutants, according to the Agency of protection of the environment (EPA) of the Government of the United States. Section 112 of the Clean Air Act provides a list of chemical compounds known as HAPs, which include monomers such as: acrylic acid, ethyl acrylate, ethylene glycol, maleic anhydride, methyl methacrylate, phthalic anhydride and styrene, which might be used in the manufacture of resins of polyester, and particularly, of unsaturated polyester resins.

Low HAPs content refers to content equal to or below 35% by weight of compounds classified as HAPs.

VOC or VOCs refers to those compounds that are classified as volatile organic compounds according to the Agency of protection of the environment (EPA) of the Government of the United States, and which are characterized to be organic compounds that participate in atmospheric photochemical reactions, with the exception of those that the EPA considers to have a negligible effect.

Low VOC content refers to content equal to or below 35% by weight of compounds classified as VOCs.

Among the advantages of the polyester resins produced using the modified oligoesters with cyclic acetals of this invention are:

that chain stoppers used in its manufacturing process are not toxic, or flammable, or hazardous for human health and the environment, in comparison with dicyclopentadiene;

in the obtaining of the modified oligoesters of the present invention, waste polyesters and glycerol derived from naturally occurring triglycerides are used, achieving a higher content of renewable material in their composition;

provides a greater flame retardancy that when flammable chain stoppers are used, especially in the case of dicyclopentadiene;

thanks to the presence of cyclic acetal groups, the oxygen inhibition is reduced during the curing of the resin, thus achieving a better curing of the surface exposed to the air;

due to the presence of cyclic acetal groups is achieved an improved compatibility with additives used as thixotropic agents, which means a reduction of up to 50% in the amount of additives required to achieve a same rate of thixotropy.

Thixotropy index refers to the relationship between the dynamic viscosity of the resin during its application and the viscosity of the resin at rest.

Another advantage of these resins is that when they are pre-accelerated through the incorporation of curing additives, the resin viscosity does not significantly change during storage for times that can reach up to 2 years.

Curing additives include, but are not limited to, the metal transition carboxylates, such as cobalt and zirconium carboxylates.

The amount of curing additives employed in the chemical composition may be between about 0.001% to about 2% of the chemical composition; preferably between about 0.005% to about 0.5% of the chemical composition; and more preferably between about 0.005% to about 0.1% of the chemical composition.

Another advantage of these resins is that they present an enhanced chemical resistance and better mechanical resistance compared with other resins manufactured using chain stoppers from the state of the art, especially with dicyclopentadiene.

Chemical resistance refers to the ability of the resin of conserving its mechanical properties when exposed to direct contact with chemicals, such as strong acids, strong bases, or water.

Mechanical strength refers to the pressure that supports a piece of material subjected to stress, either tensile or flexural, before fracture.

Another advantage of these resins, with respect to other unsaturated polyester resins, is that after being cured they present lower amounts of residual reactive solvent.

Reactive solvent refers to those compounds that are part of the reactants of a reaction, but are also able to dilute or dissolve the products of interest.

Examples of reactive solvents include but are not limited to 2-hydroxyethyl acrylate, 2-hydroxyethyl methacrylate, 2-hydroxypropyl acrylate, 2-ethylhexyl acrylate, acetoacetoxyethyl methacrylate, acrylic acid, methacrylic acid, butanediol dimethacrylate, butyl acrylate, diallyl phthalate, diethylene glycol dimethacrylate, divinyl benzene, styrene, ethyl acrylate, ethylene glycol dimethacrylate, methyl methacrylate, para-tert-butyl styrene, propylene glycol di methacrylate, triethylene glycol dimethacrylate, trimethylpropane trimethacrylate, vinyl acetate, vinyl toluene, and α-methyl styrene.

The process for the production of polyester resins using the modified oligoesters with cyclic acetals of the present invention includes the polyesterification reaction, in the presence of such oligoesters, between polyols, saturated or unsaturated polycarboxylic acids, hydroxyacids, and other compounds for the formation of the polyester such as lipids, catalysts, solvents and additives.

The amount of modified oligoesters employed in the polyester resin may be between about 0.01% to about 80% of the polyester resin; preferably between about 5% to about 65% of the polyester resin; and more preferably between about 20% to about 50% of the polyester resin.

The amount of solvent employed in the polyester resin may be between about 0.1% to about 70% of the polyester resin; preferably between about 1% to about 50% of the polyester resin; and more preferably between about 5% to about 35% of the polyester resin.

The amount of catalyst employed in the polyester resin may be between about 0.001% to about 2% of the polyester resin; preferably between about 0.01% to about 0.5% of the polyester resin; and more preferably between about 0.01% to about 0.1% of the polyester resin.

The amount of additive employed in the polyester resin may be between about 0.001% to about 2% of the polyester resin; preferably between about 0.005% to about 0.5% of the polyester resin; and more preferably between about 0.005% to about 0.1% of the polyester resin.

After the polyesterification step, a step of adjustment of the resin is performed that includes dissolving the obtained polymer in a suitable solvent, and also adding additional solvent or additives required to achieve the physicochemical and performance properties desired for the final product. For example, solvent is used to adjust the viscosity of the resin, whereas curing additives and inhibitors are used to adjust the curing time of the resin.

Lipid refers to hydrophobic organic compounds that occur naturally in living organisms.

Examples of lipids include, but are not limited to, algae oil, cottonseed oil, coconut oil, rapeseed oil, sunflower oil, castor oil, jatropha oil, linseed oil, corn oil, peanut oil, olive oil, palm oil, palm kernel oil, fish oil, soybean oil, and tung oil.

When used, the amount of lipids employed in the polyester resin may be between about 0.1% to about 30% of the polyester resin; preferably between about 1% to about 20% of the polyester resin; and more preferably between about 5% to about 15% of the polyester resin.

In the process of synthesis of polyester resins using the modified oligoesters of the present invention, usually is used a ratio of hydroxyl to carboxyl equivalents between 0.5 and 1.5, preferably between 0.8 and 1.2, and most preferably between 0.9 and 1.1. To carry out this polyesterification reaction, a temperature between 50° C. and 350° C., preferably between 100° C. and 300° C., and more preferably between 150° C. and 250° C. is used. It is additionally used a reaction pressure of usually between 10 kPa and 2 MPa, preferably between 20 kPa and 1 MPa, and most preferably between 50 kPa and 0.5 MPa.

As catalysts for the polyesterification reaction may be used organic or inorganic acids or anhydrides, organometallic acids, metal carboxylates, organometallic carboxylates, metal hydroxides, lipases, organometallic oxides, acidic or basic ion exchange resins, acidic or basic zeolites, among others. Preferably it may use as a catalyst inorganic acids compounds and organometallic acids. The amount of catalysts employed in the polyesterification is usually lower than 0.5% by weight of the total of the reagents, preferably less than 0.5% by weight of the total of the reagents, and more preferably less than 0.1% by weight of the total of the reagents. The polyesterification reaction progresses until reaching an acid value between 0 and 80 mg KOH/g resin, preferably an acid value between 10 and 60 mg KOH/g of sample and most preferably between 20 and 40 mg KOH/g of sample. Additionally, the polyesterification process may include a purification step of the polyester resin.

Acid value refers to the content of free carboxylic acids present in a polyester resin. It is expressed in terms of milligrams of potassium hydroxide required to neutralize 1 gram of resin.

Example 6 presents an unsaturated polyester resin obtained using the modified oligoesters with cyclic acetals of the present invention. In this example the modified oligoesters of Example 5 are used to obtain a very low viscosity resin (less than 0.2 Pa·s/25° C.) and a low styrene content (less than 35% by weight).

The polyester resins made by using the modified oligoesters of the present invention, can be used to obtain polymeric materials compositions such as coatings and composites, which offer a low solvent content and a low emission of the same, and at the same time ensure proper performance.

The amount of modified oligoesters employed in the chemical composition may be between about 0.01% to about 60% of the chemical composition; preferably between about 1% to about 50% of the chemical composition; and more preferably between about 10% to about 40% of the chemical composition.

In the case of coatings, the polyester resins of the present invention are employed as film former. In the case of composites, the polyester resins of the present invention are diluted in reactive solvent reagents and are used as the polymer matrix of the composite.

Coating refers to a composition that is deposited on the surface of an object, and is used to improve certain properties of this surface such as appearance, optical properties, mechanical strength, chemical resistance, resistance to corrosion, durability, among others.

Composite refers to a heterogeneous material composed of 2 or more different materials, whose properties obtained are different to those of the individual materials. Composites are usually formed by a matrix and a reinforcing agent.

Film former refers to a material which is capable of forming a uniform film after applied on the surface of an object.

Polymer matrix refers to one of the components of a composite, which includes a material having polymers that serves as a base or matrix for the composite.

One advantage of the composites which are produced using unsaturated polyester resins made from the oligoesters modified with cyclic acetals of this invention, with respect to the composites obtained from other types of unsaturated polyester resins, is that thanks to the presence of acetal groups during the preparation of the composite a greater wetting of the reinforcement fibers with the polyester resin is achieved.

Examples of reinforcing fibers include, but are not limited to, carbon fibers, natural fibers, and glass fibers.

Another advantage of these composites is that they present a lower migration of residual reactive solvent across the surface of the same, with respect to other composites.

Another advantage of these composites is that they present a greater tensile and flexural strength that may be up to 10% superior with respect to other composites.

Another advantage of these composites is that they present a greater elongation at break, which can be up to 100% superior with respect to other composites.

Another advantage of these composites is that they have a greater impact resistance with respect to other composites.

Example 7 shows a composite made from unsaturated polyester resin made from the oligoesters modified with cyclic acetals of the present invention, and fiberglass. The unsaturated polyester resin of Example 6 was used for this purpose. The composite obtained presented a hardness of 35 units in Barcol scale after 24 hours of their manufacture. After post-cure at 80° C. for 3 hours, it reached a hardness of 50 units in Barcol scale.

### EXAMPLES

### Example 1

In a glass container with mechanical agitation, equipped with a thermometer and a temperature controller, nitrogen input, reflux column, condenser, water trap, and heated by an electric mantle, were charged 1,2-propanediol, phthalic anhydride, glycerol formal (mixture of 60% by weight of 5-hydroxy-1,3-dioxane and 40% by weight of 4-hydroxymethyl-1,3-dioxolane) and dibutylstannous oxide as catalyst according to the amounts presented in Table 1.

TABLE 1
Oligoesters modified with monohydric cyclic
acetal by the esterification process

| Material | Quantity |
|---|---|
| 1,2-propanediol | 8 moles |
| Phthalic anhydride | 8 moles |
| Glycerol formal | 8 moles |
| Dibutylstannous | 0.002 moles |
| oxide | |
| Theoretical water | 8 moles |

The mixture was heated to 230° C. and a pressure of 85 kPa, with permanent vapor removal and condensation. The heating was suspended when at least 90% of the theoretical water according to Table 1 was removed, which is equivalent to an average degree of polymerization of 2.9. The obtained oligoesters have a melting point close to 105° C. One of the reactions that occur during this process, and that leads to the formation of oligoesters modified with cyclic acetals is as follows:

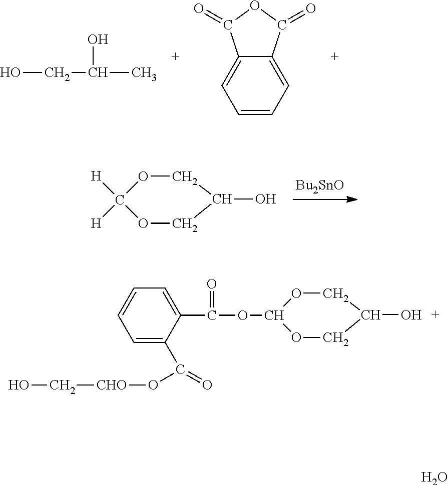

### Example 2

In a glass container with mechanical agitation, equipped with thermometer and temperature controller, nitrogen, reflux column, condenser, water trap, and heated by electric mantle, were charged waste polyethylene terephthalate (PET), oligoacetal obtained from the reaction of glycerol with formaldehyde in a molar ratio 1:1 and a zinc acetate catalyst according to the amounts presented in Table 2. The mixture was heated to 220° C. and a pressure of 85 kPa. The heating was suspended after 300 minutes, achieving a final viscosity of 26.9 Pa·s at 25° C. One of the reactions that occur during this process, and that leads to the formation of oligoesters modified with cyclic acetals is as follows:

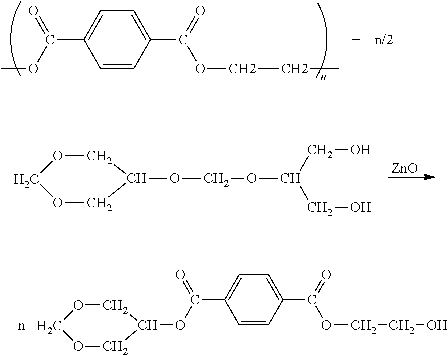

TABLE 2
Oligoester modified with oligoacetal by the process
of transesterification using waste polyester

| Material | Quantity |
|---|---|
| PET | 5 moles (as ethylene terephthalate) |
| Oligoacetal | 5 moles (as glycerol formal) |
| Zinc acetate | 0.011 moles |

### Example 3

In a glass container with mechanical agitation, equipped with thermometer and temperature controller, nitrogen, reflux column, condenser, water trap, and heated by electric mantle, were charged virgin lactic polylacid (PLA), glycerol formal and zinc acetate catalyst in accordance with the quantities presented in Table 3. The mixture is heated to 210° C. at a pressure of 85 kPa. Heating was suspended after 370 minutes, achieving a final viscosity of 46 mPa·s at 25° C. One of the reactions that occur during this process, and that leads to the formation of oligoesters modified with cyclic acetals is as follows:

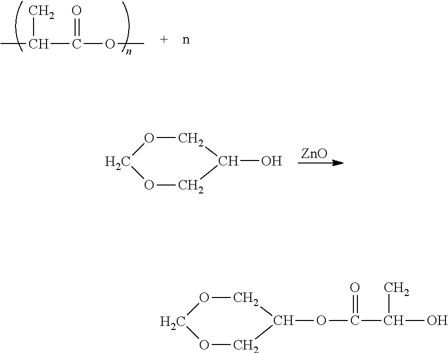

TABLE 3
Oligoesters modified with monohydric cyclic acetal by the
process of transesterification with virgin polyester

| Material | Quantity |
|---|---|
| PLA | 3.75 moles (as Lactide) |
| Glycerol formal | 15 moles |
| Zinc acetate | 0.013 moles |

### Example 4

In a glass container with mechanical agitation, equipped with thermometer and temperature controller, nitrogen, reflux column, condenser, water trap, and heated by electric mantle, were charged waste polyethylene terephthalate (PET), 2,2-dimethyl-4-hydroxymethyl-1,3-dioxolane (solketal) and zinc acetate catalyst in accordance with the quantities presented in Table 4. The mixture was heated to 220° C. and a pressure of 85 kPa. Heating was suspended after 370 minutes, achieving a final viscosity of 1.5 Pa·s at 25° C. One of the reactions that occur during this process, and that leads to the formation of oligoesters modified with cyclic acetals is as follows:

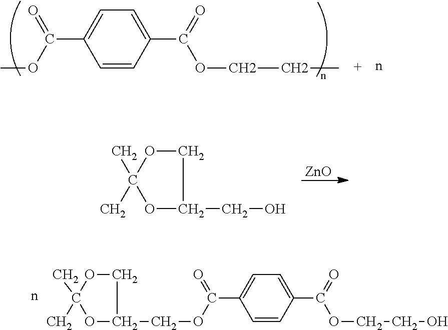

TABLE 4
Oligoesters modified with monohydric cyclic acetal by
the process of transesterification with waste polyester

| Material | Quantity |
|---|---|
| PET | 4.5 moles (as ethylene terephthalate) |
| Solketal | 9 moles |
| Zinc acetate | 0.013 moles |

### Example 5

In a glass container with mechanical agitation, equipped with thermometer and temperature controller, nitrogen, reflux column, condenser, water trap, and heated by electric mantle, were charged waste polyethylene terephthalate (PET), virgin lactic polyacid (PLA), glycerol formal, and zinc acetate catalyst in accordance with the quantities presented in Table 5. The mixture was heated to 200° C. at a pressure of 85 kPa. Heating was suspended after 250 minutes, achieving a final viscosity of 0.54 Pa·s at 25° C. One of the reactions that occur during this process, and that leads to the formation of oligoesters modified with cyclic acetals is as follows:

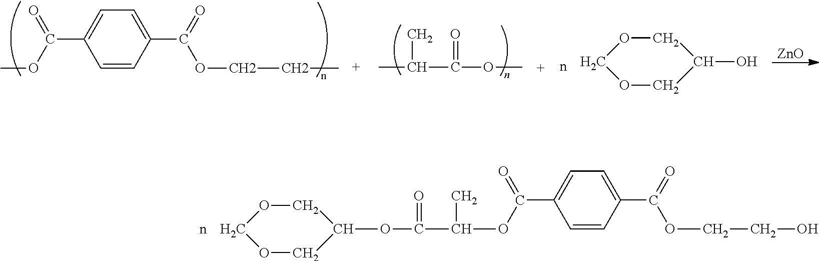

TABLE 5
Oligoesters modified with monohydric cyclic acetal
by the process of transesterification with mixture
of virgin polyester and waste polyester

| Material | Quantity |
|---|---|
| PET | 0.55 moles (as ethylene terephthalate) |
| PLA | 0.73 moles (as Lactide) |
| Glycerol formal | 3.18 moles |
| Zinc acetate | 0.001 moles |

### Example 6

In a glass container with mechanical stirring, equipped with thermometer and temperature controller, nitrogen input, reflux column, condenser, water trap, and heated by electric mantle, were charged the modified oligoesters of example 5, maleic anhydride, and phthalic anhydride in accordance with the quantities presented in Table 6. The mixture was heated to 215° C. at a pressure of 85 kPa, with permanent removal and condensation of vapors. Heating was suspended when it reaches an acid value of less than 35 mg KOH/g resin. The mixture was cooled to 170° C. and hydroquinone was added according to Table 6. The mixture was cooled down to 130° C. and styrene, toluhydroquinone and parabenzoquinone were added in the amounts corresponding to those presented in Table 6.

The final resin viscosity is 125 mPa·s, for a content of styrene of 33.3% weight.

TABLE 6
Polyester resin manufactured from oligoesters
modified with cyclic acetals of example 5

| Material | Quantity | |
|---|---|---|
| Example 5 | 542 | grams |
| Maleic anhydride | 120 | grams |
| Phthalic anhydride | 18 | grams |
| Hydroquinone | 0.09 | grams |
| Styrene | 334 | grams |
| Toluhydroquinone | 0.37 | grams |
| Parabenzoquinone | 0.02 | grams |

### Example 7

Carnauba wax was applied on a glass mold. Two squares of a fiberglass mat were cut having a density of 450 g/m² with a weight of 45 grams each. 210 grams of the polyester resin of Example 6 were weighted and 4.2 grams of methyl ethyl ketone peroxide 9% of active oxygen were added. The catalyzed resin was poured over the mold until it was completely covered with resin. One of the squares of glass fiber was placed over the resin and with using a metal roller and a brush the resin was incorporated into the fiberglass. When the fiber layer was completely wetted by resin, the second fiberglass layer was placed and the rest of the catalyzed resin was added, pressing with the roller to distribute the resin evenly over the fiberglass.

The composite thus obtained was left to cure at room temperature for 24 hours and then was removed from the mold and the ends were polished. Then the material was post cured in an oven at 80° C. for 3 hours, to achieve a maximum development of mechanical properties. 5 test specimens according to the standard ASTM D638 were prepared and the mechanical properties of the composite subjected to tensile stress were evaluated. The results obtained are summarized in Table 7.

TABLE 7
Mechanical properties of the composite retrieved using
the example 6 polyester resin manufactured from oligoesters
modified with cyclic acetals of Example 5

| Property | Result | |
|---|---|---|
| Hardness before post - cure | 35 | Barcol |
| Hardness after post - cure | 50 | Barcol |
| Young modulus (tensile) | 5.85 | GPa |
| Deformation at maximum load | 2.14% | |
| (tensile) | | |
| Maximum effort (tensile) | 97.35 | MPa |

1. A modified oligoester comprising:
an oligoester (1) containing at least one esterifiable functional group in one terminations and bounded in one or more of the ends of a chain with a cyclic acetal group (2).

2. The modified oligoester according to claim 1, wherein the modified oligoester has a formula: 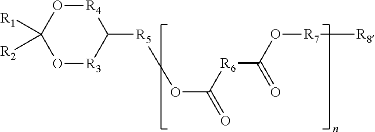
where:
R₁ and R₂ independently represent organic groups in general, and hydrogen;
R₃, R₄, and R₅ represent (CH₂)ₐ groups, with whole integer values of “a” greater or equal to zero, independent for each group;
R₆ and R₇ independently represent organic groups;
R₈ represents a group that forms covalent bonds with —OH groups or —COOH groups, selected from the group consisting of alcohols (6), carboxylic acids (7), aldehydes (8), ketones (9), amines (10), acyl halides (11), inorganic acids (12), and hydroxides (13);
“n” is a positive number greater than or equal to 1 and less than or equal to 10.

3. The modified oligoester according to claim 1, wherein the oligoester (1) comes from:
an esterification reaction between esterifiables monomers selected from the group consisting of polyols (3), carboxylic polyacids (4), and hydroxyacids (5),
or
depolymerization of virgin or waste polyesters, including at least one of the following:
polyglycolic acid, polylactic acid, polycaprolactone, polyethylene adipate, polyethylene naphthalate, polyethylene terephthalate, polybutylene succinate, polybutylene terephthalate, polytrimethylene terephthalate, polyhydroxybutyrate, and polyhydroxyvalerate.

4. The modified oligoester according to claim 1, wherein the cyclic acetals (2) groups come from oligoacetals (15) or monohydric cyclic acetals (17).

5. The modified oligoester modified according to claim 4, wherein the oligoacetals (15) come from the reaction between a triol (16) with an aldehyde (8) or a ketone (9).

6. The modified oligoester according to claim 4, wherein the monohydric cyclic acetals (17) come from the reaction between a triol (16) with an (8) aldehyde or a ketone (9).

7. The modified oligoester modified according to claim 4, wherein the monohydric cyclic acetals (17) come from reaction between glycerol (18) with an aldehyde (8) or a ketone (9).

8. A process to obtain the modified oligoester of the claim 1, the process comprising the steps of:
(a) synthesizing an oligoester (1) via the depolymerization of polyesters or a reaction between esterifiables monomers selected from the group consisting of polyols (3), carboxylic polyacids (4), and hydroxyacids (5);
(b) reacting the oligoester (1) of the step (a) and a compound containing one or more cyclic acetal groups and one or more alcohol groups.

9. The process for obtaining modified oligoester according to claim 8, wherein the compound containing acetals groups and the hydroxyl groups of the step (b) are selected from the group comprising monohydric cyclic acetals (17) and oligoacetals (15).

10. The process for obtaining modified oligoester according to claim 8, wherein the reaction temperature is between about 100 to about 300° C. and with a pressure of between about 20 kPa and about 2 MPa.

11. The process for obtaining modified oligoester according to claim 8, wherein a degree of polymerization is between about 2 and about 10.

12. The process for obtaining modified oligoester according to claim 8, at further including the step of purification of the modified oligoester resulting from step (b).

13. A chemical composition comprising the modified oligoester according to claim 1, at least one compound selected from the group consisting of hydroxy acids (5), polyols (3) and carboxylic polyacids (4), and at least one compound selected from the group consisting of catalysts (19), solvents (20), and additives (21).

14. The chemical composition according to claim 13 wherein the chemical composition is a polyester resin.

15. The chemical composition according to claim 14, wherein the polyester resin contains dicyclopentadiene.

16. The chemical composition according to claim 14, wherein the polyester resin includes a solvent content of less than 35% by weight.

17. The chemical composition according to claim 16, wherein the solvent is styrene in an amount between 0% and 35% by weight.

18. The chemical composition according to claim 16, wherein the polyester resin has a content of compounds classified as HAPS equal to or less than 35% by weight and a content of compounds classified as VOCs equal to or less than 35% by weight.

19. The chemical composition according to claim 14, wherein the chemical composition is a coating composition containing a polyester resin as film former.

20. The chemical composition according to claim 14, wherein the chemical composition is a composite including a polyester resin as a polymeric matrix.

21. A process for obtaining the polyester resin according to claim 14, further including the steps of:
(c) polymerization in the presence of the modified oligoester of claim 1, two or more monomers selected group comprising hydroxy acids (5), polyols (3), carboxylic polyacids (4);
(d) adjusting the polyester obtained in step (a)

22. The process for the production of resin polyester according to claim 21, wherein the polymerization step (a) is performed in the presence of lipids (22), catalysts (19), solvents (20) and additives (21).

23. The process for the production of resin polyester according to claim 21, wherein the reaction of the step (a) the temperature is between about 100 to about 300° C. and the pressure is between about 20 kPa and about 2 MPa.

24. The process for the production of resin polyester according to claim 21, wherein in the polymerization step (a) includes an esterification process, a transesterification process, or combination thereof.

25. The process for the production of resin polyester according to claim 21, wherein the reaction has a final acid value preferably between about 10 and about 60 mg KOH/g resin.

26. The process for the production of resin polyester according to claim 21, further including a purification step for the resin.

27. The use of the modified oligoester of claim 1, as a chain stopper for the manufacture of polyester resins.
